(19) United States
(12) Patent Application Publication
(10) Pub. No.: US 2024/0382323 A1
(43) Pub. Date: Nov. 21, 2024

(54) PROSTHETIC SYSTEM

(71) Applicant: Open Bionics Ltd, Bristol (GB)

(72) Inventors: Steve Wood, Swindon Wiltshire (GB); Jonathan Raines, Bristol (GB)

(73) Assignee: Open Bionics Ltd, Bristol (GB)

(21) Appl. No.: 18/664,724

(22) Filed: May 15, 2024

(30) Foreign Application Priority Data
May 17, 2023 (GB) 2307355.4

(51) Int. Cl. A61F 2/80; A61F 2/54

(52) U.S. Cl. CPC A61F 2/80; A61F 2/54

(57) ABSTRACT

A prosthetic system includes an inner socket configured to receive a residual limb, the inner socket having at least one socket window, and an outer frame configured to receive the socket, the frame having at least one frame window. The socket window is configured to align with the frame window when the socket is received within the frame. The frame window and the socket window are together configured to releasably receive a sensor for the residual limb.

### BACKGROUND OF THE INVENTION

The present disclosure relates generally to a prosthetic system and particularly, but not exclusively, to a prosthetic system including an inner socket and an outer frame.

The upfront costs associated with providing an amputee (or a person with a congenital limb deficiency) with a prosthetic system can be expensive. Consumers looking to buy a prosthetic system for personal use and organizations (such as state healthcare providers or insurance companies) that supply prosthetic systems to users may, therefore, incur high costs in purchasing and fitting prosthetic systems that may not be recuperated if the user subsequently rejects the product. The prosthetic system may further have been made bespoke to the user, which may pose complications in repurposing or recycling the prosthetic system to the needs of a different user.

It is thus desirable to develop a prosthetic system having a modular design with components that can be readily interchanged and replaced, for example, by the supplier of the prosthetic system, the end consumer, or during assembly. This can improve a possibility of repurposing a rejected prosthetic system. This can also give a consumer or supplier a lower cost option for replacing a broken or faulty component of a prosthetic system.

Furthermore, the residual limb of the user may change in volume (and, therefore, shape) over the course of a day. This may occur, for example, if the user gets warmer or cooler. The prosthetic system may not adapt to the changing volume of the residual limb of the user, which can cause user discomfort. It is, therefore, also desirable to develop a prosthetic system having an improved adjustability and ventilation, which may reduce user discomfort and consequently reduce a likelihood of the prosthetic system being rejected by the user.

### SUMMARY OF THE INVENTION

According to a specific aspect, there is provided a prosthetic system including an inner socket configured to receive a residual limb and an outer frame configured to receive the socket. The socket may include at least one socket window. The frame may include at least one frame window. The socket window may be configured to align with the frame window when the socket is received within the frame. The socket window and the frame window may be together configured to releasably receive a sensor for the residual limb.

The socket may include an inner surface and an outer surface. The inner surface may be configured to be adjacent to the residual limb. The outer surface of the socket may be configured to be adjacent to an inner surface of the frame. The one or more socket windows may be formed between the inner surface of the socket and the outer surface of the socket. The one or more frame windows may be formed between the inner surface of the frame and an outer surface of the frame.

The socket window may include at least one window tooth configured to engage (e.g., resiliently engage) a corresponding sensor tooth when the sensor is placed into the socket window.

Alternatively, the socket window may include any appropriate means configured to engage a corresponding sensor.

The frame window may include at least one window tooth configured to engage (e.g., resiliently engage) a corresponding sensor tooth when the sensor is placed into the frame window.

The socket window and/or frame window may include a plurality of teeth. The space between adjacent window teeth may be configured to provide a snap-fit for a sensor tooth. The sensor tooth may be configured to move in sequential snap-fits between adjacent window teeth when a user exerts a force on the sensor.

The window teeth may be equally spaced. The window teeth may be formed between an edge of the socket window adjacent to the outer surface of the socket and an edge of the socket window adjacent to the inner surface of the socket. As such, pushing or pulling the sensor may enable the sensor to be adjusted closer to the inner surface of the socket or further away from the inner surface of the socket in sequential snap-fits.

The prosthetic system may further include the sensor. The sensor may include at least one tooth for engaging the window tooth (e.g. of the frame window and/or sensor window).

The window teeth and/or sensor teeth may be arranged in a saw-tooth pattern.

The sensor may sense a biomedical parameter of a wearer of the prosthetic system. The sensor may be an electromyography sensor, e.g., to detect muscle response or electrical activity in response to a nerve's stimulation.

The socket may include a proximal end and a distal end, and the proximal end may include an open portion configured to allow a residual limb to be inserted into the socket or removed from the socket.

The socket may include a socket slit. The socket slit may extend from an edge of the open portion and substantially toward the distal end of the socket. The width of the socket slit at the edge of the open portion may be configured to change relative to the width of the socket slit at the end of the socket slit that substantially approaches the distal end of the socket. This may permit the socket to adjust to a changing shape/volume of the residual limb of the user.

The socket may include a tongue. The tongue may extend along the length of the socket slit, e.g., extending across a gap defined by the slit. The tongue may be configured to be disposed adjacent to the residual limb.

The frame may include one or more channels configured to receive a cable, and the frame may further include an engagement means, e.g., hole, configured to receive a cable tensioning device.

The prosthetic system may include a cable and a cable tensioning device. The cable may be coupled to the cable tensioning device, wherein operating the cable tensioning device in a first direction may urge the frame to tighten about the socket and operating the cable tensioning device in a second direction may urge the frame to loosen about the socket.

The frame may include a frame proximal end and a frame distal end, and the frame proximal end may include a frame open portion configured to allow a residual limb to be inserted into the socket or removed from the socket.

The frame may include a frame slit. The frame slit may extend from an edge of the frame open portion and substantially towards the frame distal end. The frame slit may be configured to be disposed adjacent to (e.g., align with) the socket slit when the socket is received within the frame.

The frame proximal end may include a gap, the gap may be configured to narrow when the cable tensioning device is operated in the first direction, the gap may be configured to widen when the cable tensioning device is operated in the second direction.

The frame may include a triangular mesh.

The socket may be made from a substantially flexible and breathable material, such as thermoplastic polyurethane.

The socket may be configured to receive a residual arm limb.

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a perspective view of a prosthetic system according to an example of the present disclosure and shows a socket including socket windows disposed within a frame including frame windows such that the socket windows and the frame windows are aligned.

FIG. 2 is a perspective view of a socket according to an example of the present disclosure.

FIG. 3 is a detailed view of a socket according to an example of the present disclosure and shows a slit and a tongue of the socket.

FIG. 4 is a detailed view of a socket window according to an example of the present disclosure.

FIG. 5 is a detailed view of a sensor according to an example of the present disclosure.

FIGS. 6a and 6b (collectively FIG. 6) are section views of a socket according to an example of the present disclosure and show sensors in a neutral position and sensors in an adjusted position respectively.

FIG. 7 is a perspective view of a frame according to an example of the present disclosure.

FIG. 8 is a perspective view of a prosthetic system according to an example of the present disclosure and shows a socket including socket windows disposed within a frame including frame windows, such that the socket windows and the frame windows are aligned with a sensor inserted in each socket window.

### DETAILED DESCRIPTION OF THE PREFERRED EMBODIMENTS

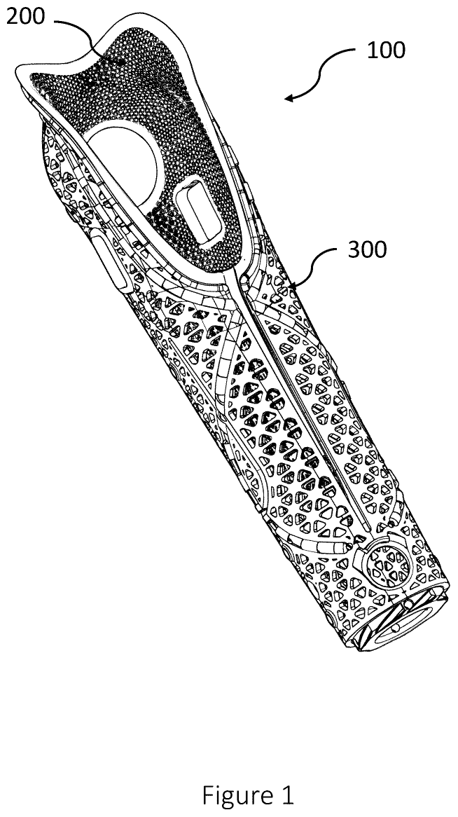

With reference to FIG. 1, a prosthetic system 100 according to an example of the present disclosure includes an inner socket 200 and an outer frame 300. The inner socket 200 is configured to receive a residual limb. The outer frame 300 is configured to receive the socket 200. For example, a user (the user may be an amputee or a person with a congenital limb deficiency) may dispose their residual limb within the socket 200. They may then dispose the socket 200 (with their residual limb disposed within the socket) within the frame 300. Alternatively, the user may first dispose the inner socket 200 within the outer frame 300. The user may subsequently dispose their residual limb within the socket 200 of the prosthetic system 100. In the present disclosure, distal refers to a point which is spaced further apart from the body of the user of the prosthetic system 100 relative to a proximal point.

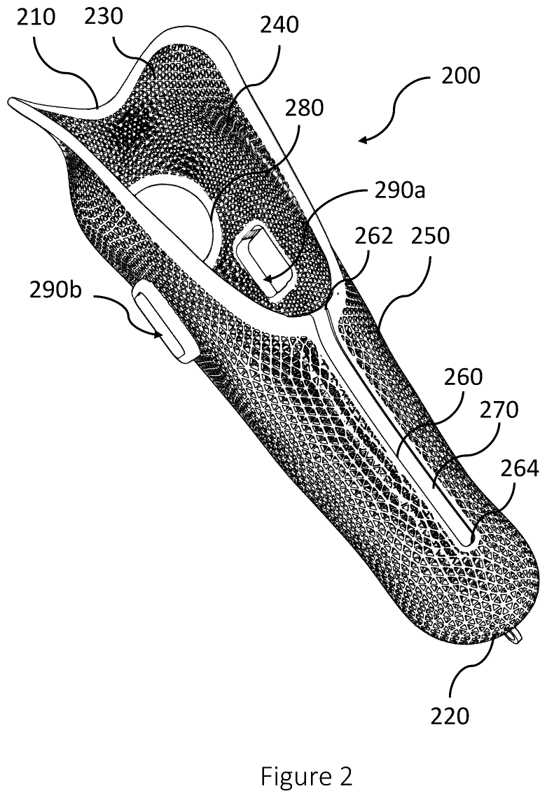

Referring now to FIG. 2, the socket 200 includes a proximal end 210 and a distal end 220. The proximal end of the socket 210 may include an open portion 230 configured to permit a user to insert or remove a residual limb from the socket 200. The open portion 230 may partially extend along the length of the socket 210. As such, the open portion 230 may include a proximal end (a proximal end of the open portion 230) and a distal end (a distal end of the open portion 230).

The distal end of the socket 220 is configured to be disposed over the distal end of the residual limb. As such, the user may insert a residual limb to the socket 200 via the open portion 230. The user may then move the socket 200 relative to the residual limb (e.g., the user may slide the socket up their residual limb) until the distal end of the user's residual limb is substantially adjacent to the distal end of the socket 220. The socket 200 may have a plurality of longitudinal flutes. The socket 200 may, therefore, have a generally cylindrical and “concertina-like” configuration. This allows, for example, the socket 200 to be expandable and compressible. Vent holes may be formed in the flutes.

As shown in FIG. 2, a width of the distal end of the socket 220 may be smaller relative to a width of the proximal end of the socket 210. The socket 200 may be configured so that there is a clearance between the distal end of the user's residual limb and the distal end of the socket 220. Alternatively, there may be a snug fit at the distal end.

Still referring to FIG. 2, the socket 200 may include an inner surface 240 and an outer surface 250. The inner surface of the socket 240 is configured to be disposed adjacent to the residual limb of the user. A substantial portion of the inner surface of the socket 240 is configured to be in direct contact with a surface of the residual limb of the user that is received by the socket 200. With the exception of (as mentioned above) a lengthwise clearance that may exist between the distal end of the user's residual limb and the inner surface 240 at the distal end of the socket 220.

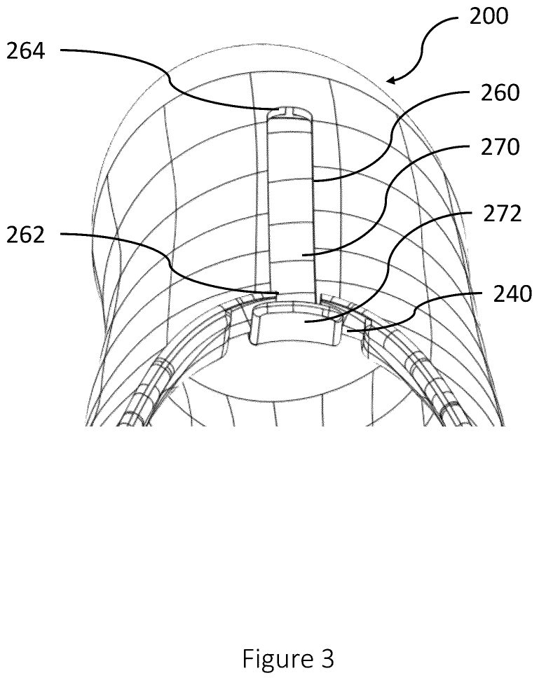

As shown in FIG. 2 and in FIG. 3, the socket 200 may include a slit 260. The slit 260 may run from a distal end of the open portion 230 and substantially toward the distal end of the socket 220. The end of the slit at the distal end of the open portion is configured to be proximal to the user wearing the socket 200 and may thus be termed the proximal slit end 262. The end of the slit substantially approaching the distal end of the socket 220 is configured to be distal to the user relative to the proximal slit end 262 when the user is wearing the socket 200 and may thus be termed the distal slit end 264. The socket may experience internal or external forces which seek to increase an internal volume of the socket 200 (e.g., the volume enclosed by the inner surface 240 of the socket 200) or decrease the internal volume of the socket 200. For example, on a warm day the residual limb of a user may expand. The socket 200 may therefore experience an internal force (from the expanded residual limb of the user) that causes the internal volume of the socket 200 to increase. The proximal slit end 262 is configured to change in width relative to the distal slit end 264. As such, the internal force experienced by the socket 200 may cause the proximal slit end 262 to increase in width relative to the distal slit end 264. The proximal slit end 262 may thus increase in width relative to the distal slit end 264. The slit 260 would therefore taper in width, increasing from the distal slit end 264 to the proximal slit end 262. As a consequence, a width of the open portion 230 of the socket 200 also increases. On the other hand, a compressive force that urges a reduction of the internal volume of the socket 200 (such as a tightening force being applied onto the frame 300 of the prosthetic system 100) may cause the proximal slit end 262 to decrease in width relative to the distal slit end 264. As a consequence, a width of the open portion 230 of the socket may decrease. Including the slit 260 may, therefore, improve an ability of the socket 200 to adjust to a changing shape/volume of the residual limb of the user. Including the slit 260 may further improve the ability of a user to manually tighten or loosen the socket 200 about the residual limb inserted therein.

The proximal slit end 262 may be bias to returning to a width that is substantially equal to the width of the distal slit end 264. The socket 200 may therefore be better suited to adapting to fluctuations in the shape/volume of the residual limb of the user. For example, if a residual limb of a user increases in shape/volume and then decreases in shape/volume, the proximal slit end 262 may first increase in width relative to the distal slit end 264, thereby causing a width of the socket 200 to widen to accommodate the increased volume of the user's residual limb. The bias of the proximal slit end 262 to return to a width that is substantially equal to the width of the distal slit end 264 may then enable the proximal slit end 262 to decrease in width relative to the distal slit end 264 as the user's residual limb decreases in volume.

An increase/decrease in width of the proximal slit end 262 relative to the distal slit end 264 is accompanied by a corresponding increase/decrease in width of the open portion 230 of the socket. The user may hence experience less discomfort as the socket is able to expand/compress in response to a fluctuation (e.g., an increase or a decrease) in the shape/volume of the residual limb inserted therein.

As is also shown in FIG. 2 and in FIG. 3, the socket 200 may include a tongue 270. The tongue 270 may run the length of the slit 260. As is best shown in FIG. 3, the tongue 270 may be adjacent to the slit 260. The tongue 270 may also be disposed adjacent to the inner surface 240 of the socket 200. As such, the tongue 270 is configured to be disposed between the inner surface 240 of the socket 200 and the residual limb of the user. A tongue inner surface 272 is thus configured to be in direct contact with the residual limb of the user. The tongue 270 may be coupled to a portion of the inner surface 240 of the socket 200 that substantially approaches the distal end 220 of the socket. The socket 200 may be manufactured by an additive manufacturing process. For example, the socket may be 3D printed. More specifically, the socket 200 may be manufactured using a Multi Jet Fusion 3D printing process. This enables the socket 200 and the tongue 270 to manufactured as one piece. The socket 200 may alternatively be manufactured using any other suitable molding process (e.g., plastic injection molding). The tongue 270 may alternatively be manufactured as a separate piece from the socket 200. The tongue may subsequently be coupled to the socket using any suitable coupling mechanism (e.g., fastenings, adhesive bonding, etc.). The width of the tongue 270 may be larger relative to the width of the slit 260 when the width of the proximal slit end 262 is substantially equal to the width of the distal slit end 264. The width of the tongue 270 may be larger relative to the width of the slit 260 when the width of the proximal slit end 262 is substantially equal to the width of the distal slit end 264. As such, the tongue 270 may reduce a likelihood of skin of a residual limb being pinched by the slit 260 when the socket 200 is tightened by a user. The tongue 270 may additionally prevent direct contact between the residual limb of the user and the frame 300 as might occur, for example, when the residual limb of the user increases in shape/volume and a width of the proximal slit end 262 increases relative to a width of a distal slit end 264.

As is best shown in the FIG. 2, the socket 200 may include a socket through hole 280. The socket through hole 280 may be disposed substantially toward the proximal end 210 of the socket 200. The socket through hole 280 may be configured to be disposed adjacent to an underside of a residual limb of the user. The socket through hole 280 may be of a width that substantially approaches a width of the open portion 230 of the socket 200. Although the socket through hole 280 is shown to be circular in the example of the present disclosure, it should be noted that it may be any shape (rectangular, triangular, oval, etc.). The socket through hole 280 may improve an airflow and ventilation of a residual limb inserted into the socket 200. Further, the socket through hole 280 may be disposed adjacent to the elbow of the user, which may improve a range of motion for the elbow of the user.

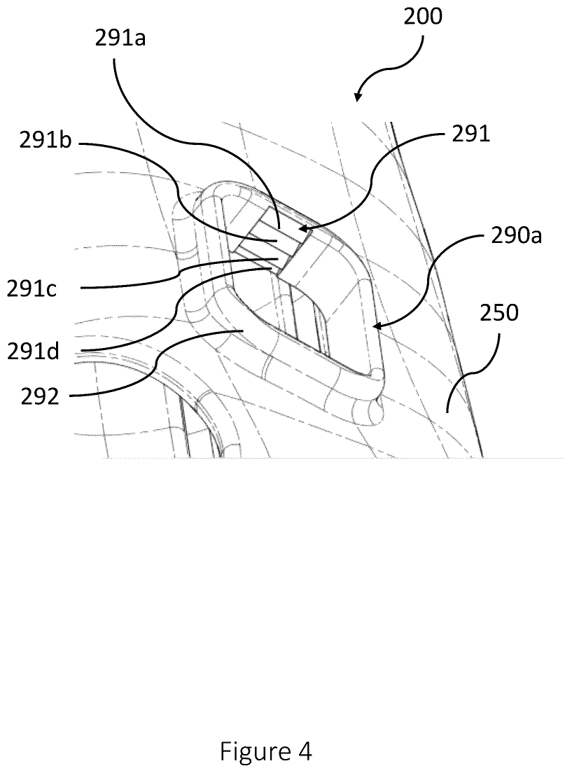

As shown in FIG. 2 and in FIG. 4, the socket 200 includes socket windows 290a, 290b. Although two socket windows 290a, 290b are shown in the example of the present disclosure, it should be noted that the socket may include one or more socket windows 290. The socket 200 may include one socket window, two socket windows, or a plurality of socket windows 290. Each socket window 290a, 290b may be a hole formed through the socket 200. Each socket window 290a, 290b may be substantially rectangular. The socket windows 290a, 290b may alternatively be any other suitable shape. The socket windows 290a, 290b may be disposed towards the proximal end 210 of the socket 200. The socket windows 290a, 290b may further be disposed between the proximal slit end 262 and the proximal end 210 of the socket. The socket windows 290a, 290b may be formed on either side of a central lengthwise line of the socket 200. The socket windows 290a, 290b may each be equally spaced from the central lengthwise line of the socket 200. As such, the socket windows 290a, 290b may be configured to be disposed on either side of a residual limb inserted into the socket 200. Alternatively, the socket windows 290a, 290b may be disposed anywhere on the socket 200. As best shown in FIG. 4, each socket window 290a, 290b may include a lip 292. The lip 292 may be formed about an edge of the socket window that is adjacent to the outer surface 250 of the socket 200. The lip 292 may be configured to mate with a corresponding window formed in the frame 300.

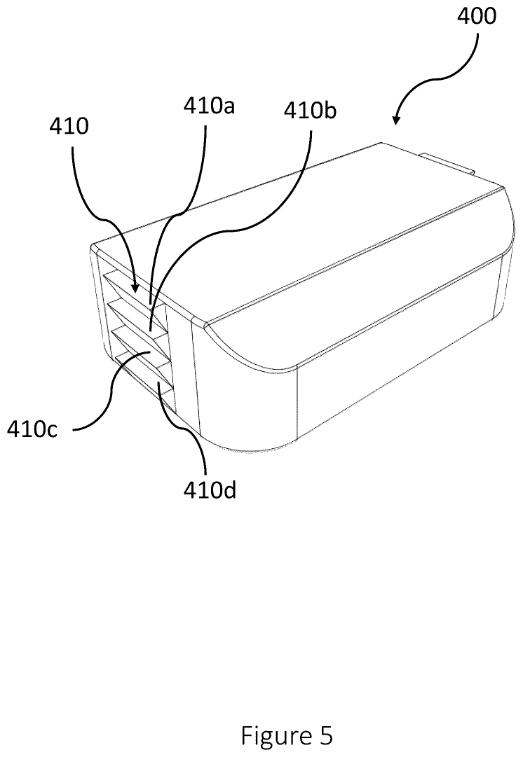

Referring now to FIG. 4 and to FIG. 5, each socket window 290a, 290b may be configured to receive a sensor 400. Each socket window 290a, 290b may include socket window teeth 291 configured to engage with sensor teeth 410. The socket window teeth 291 may include a plurality of teeth 291a-291d. Further, the sensor 400 may include a plurality of sensor teeth 410a-410d. Each sensor tooth 410a-410d is configured to snap-fit into the space formed between adjacent window teeth 291. In other words, opposing teeth may ride over one another as the sensor is pushed closer to the residual limb. The teeth may resiliently deform to provide the snap-fit function. As such, a sensor tooth 410a-410d may move in sequential snap-fits between adjacent window teeth 291. The window teeth 291 may be formed between an edge of the socket window adjacent to the outer surface 250 of the socket and an edge of the socket window adjacent to the inner surface 240 of the socket. The teeth may be arranged, e.g., in a saw-tooth fashion, to only permit movement of the sensor towards the residual limb. An interaction between opposing teeth may prevent movement of the sensor away from the residual limb. As such, the sensor 400 may be adjusted closer to the inner surface 240 of the socket 200 in sequential snap-fits. The teeth may be arranged to also permit movement of the sensor 400 further away from the inner surface 240 of the socket. This enables a user to adjust the proximity of the sensor 400 relative to a residual limb inserted to the socket. Additionally, the user is permitted to remove the sensor 400 by pushing the sensor 400 all the way through the socket window 290a, 290b. For example, the user may remove their residual limb from the socket 200 and then remove the sensor 400 by pushing the sensor 400 all the way through the socket window 290a, 290b. This may provide an ergonomic advantage to the prosthetic system because a user may easily remove or otherwise adjust the sensor 400 when it is in the socket window 290a, 290b.

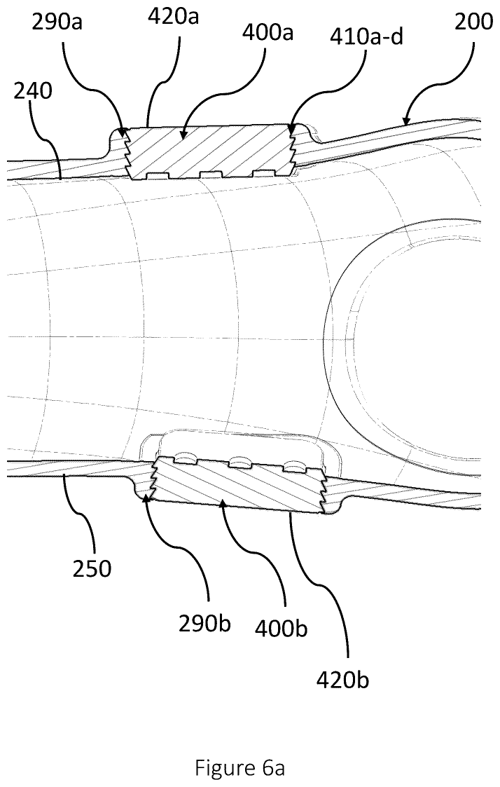

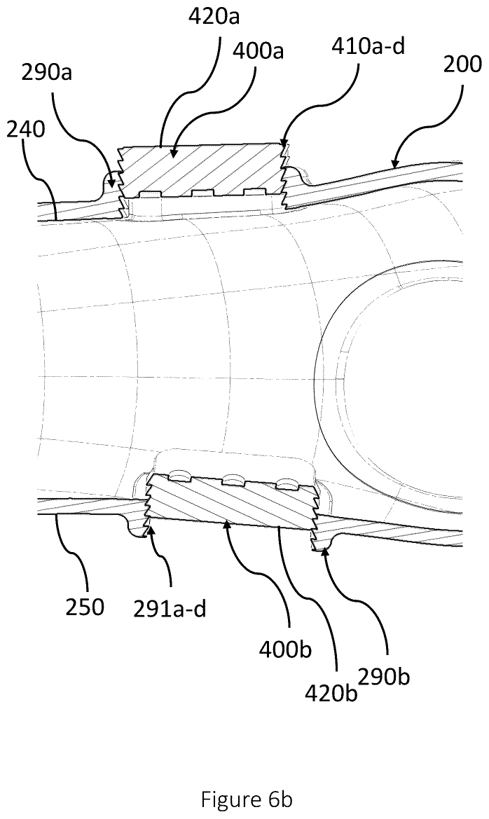

Adjustment of the sensors 400 is shown through FIG. 6a to FIG. 6b. FIG. 6a shows sensor 400a and sensor 400b inserted into socket windows 290a and 290b respectively. The sensors 400a, 400b may be considered to be in a neutral position, in which an outer surface 420a, 420b of each sensor is substantially flush to the outer surface 250 of the socket 200. In the neutral position, each sensor tooth 410a-410d has respectively been snap-fit into a space formed between adjacent window teeth 291a-291d. FIG. 6b shows an adjustment of sensor 400a and sensor 400b relative to the position of the sensors as shown in FIG. 6a. Sensor 400a has been adjusted such that the outer surface 420a of the sensor 400a has been further spaced apart from the inner surface 240 of the socket 200. Sensor 400b has been adjusted such that the outer surface 420b of the sensor 400b has been spaced closer to the inner surface 240 of the socket 200.

Although the example of the present disclosure shows four sensor teeth 410a-410d on either side of the sensor, it is noted that the sensor 400 may include any number of teeth. For example, the sensor 400 may include one tooth, the tooth being configured to move in sequential snap-fits between adjacent window teeth 291. It is further noted that although the example of the present disclosure shows four window teeth 291a-291d on either side of the socket window 290a, 290b, the socket window may include any number of teeth. For example, the socket window 290a, 290b may include one window tooth, the tooth being configured to move in sequential snap-fits between adjacent sensor teeth 410. The number of window teeth 291 may be configurable in manufacture, wherein increasing the number of window teeth 291 may increase the number of sensor 400 adjustment positions but may also increase manufacturing time and complexity. The number of sensor teeth 410 may be configurable in manufacture, wherein increasing the number of sensor teeth 410 may increase the number of sensor 400 adjustment positions but may also increase manufacturing time and complexity.

The example of the present disclosure shows window teeth 291 and sensor teeth 410 as the engagement means between the sensor 400 and the socket window 290. It is noted however that any means for enabling the sensor 400 to be placed in the socket window 290 may be applied.

The socket windows 290a, 290b and the window teeth 291 may be included in the computer-aided design (CAD) model of the socket 200 for 3D printing. As such, the socket windows 290a, 290b (including the window teeth 291) and the socket may be manufactured as one piece. The socket windows 290a, 290b (including the window teeth) may alternatively be manufactured separately from the socket 200. For example, the socket windows 290a, 290b (including the window teeth 291) may be 3D printed or plastic injection molded (or any other suitable process) separately to the socket 200 and then coupled (e.g., slotted into and adhesively bonded or fastened) to a corresponding socket-window-through-hole formed in the socket 200.

The socket 200 and the socket windows 290 may be manufactured from a thermoplastic polyurethane (TPU). The TPU may be a TPU suitable for 3D printing. The socket windows 290 may be manufactured from any suitable material (e.g., a suitable TPU) that is rigid relative to the material used to manufacture the socket 200. This may improve an ease of use of the prosthetic system 100, as the rigidity of the socket window 290 relative to the socket 200 may improve an ability of a user to insert a sensor 400 to the socket window 290, remove the sensor 400 from the socket window 290, and adjust a sensor 400 in the socket window 290. This may further improve a comfort and adjustability of the socket 200, as the socket 200 may be made from a flexible material that adjusts to a changing shape/volume of a residual limb of a user.

In some embodiments, the present invention provides or relates to a myoelectric prosthetic system that uses the electrical tension generated every time a muscle contracts as information. The sensor 400 may be an electromyography (EMG) sensor. The EMG sensor may be configured to be in direct contact with the residual limb of the user. More specifically, the EMG sensor may be configured to be in direct contact with a portion of skin of the residual limb of the user. The EMG sensor may further be configured to detect a voltage or electrical tension relating to contractions of a specific muscle or a specific group of muscles in the residual limb of the user. The EMG sensor may thus be configured to detect an electrical activity of the muscle (or muscles). The EMG sensor may therefore be configured to send a signal to a controller upon detection of an electrical activity within the muscle. The controller may be configured to control an operation of a prosthetic hand (including finger movement, wrist rotation, etc.) based on the sensed electrical activity. Although the example of the present disclosure is concerned with an EMG sensor, it is noted that the sensor 400 may be any sensor. It is further noted that the sensor 400 may be a wireless sensor, e.g., one that may communicate wirelessly with a controller. The sensor may communicate via Bluetooth or any other wireless protocol. As such, cables for connecting to the sensor 400 may not be embedded into the socket 200 or the frame 300.

In some embodiments, the present invention provides a transradial prosthesis—an artificial limb that replaces an arm missing below the elbow. In other embodiments, the present invention provides a transhumeral prosthesis-a prosthetic lower and upper arm, including a prosthetic elbow. Further, although the residual limb may be considered to be a residual arm limb of a user, the prosthetic system may be applied to other residual limbs, such as a residual leg limb. In this second example, the controller may be configured to control an operation of a prosthetic foot (including toe movement, ankle rotation, etc.).

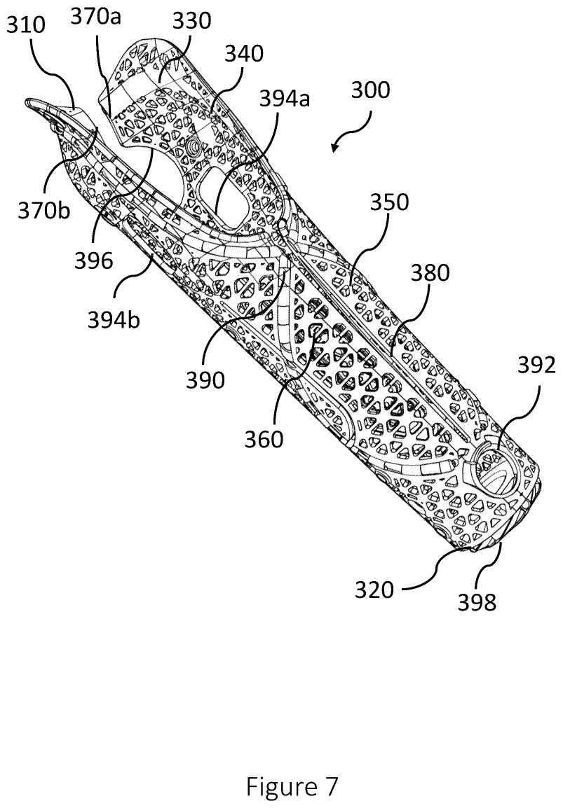

As best shown in FIG. 7, the frame 300 may include a proximal end 310 and a distal end 320. The proximal end 310 of the frame 300 may include an open portion 330 configured to permit a user to insert or remove the residual limb from the socket 200. The open portion 330 may partially extend along the length of the frame 300. As such, the open portion 330 may include a proximal end (a proximal end of the open portion 330) and a distal end (a distal end of the open portion 330).

Referring to FIG. 2 and FIG. 7, the outer surface 250 of the socket 200 may be configured to be disposed adjacent to an inner surface 340 of the frame 300. The profile of the inner surface 340 of the frame 300 may be substantially similar to the profile of the outer surface 250 of the socket 200. Consequently, a contact area between the socket outer surface 250 and the frame inner surface 340 may be maximized.

The outer frame 300 may have an open core lattice structure. This provides strength while at the same time inherently providing ventilation. The frame 300 may include an outer surface 350. The frame 300 may include a plurality of polygon extrusions 360 formed between the inner surface 340 and the outer surface 350 of the frame 300. The polygon extrusions 360 may be triangular. As such, the frame 300 may be considered to include a triangular mesh. The polygon extrusions 360 may permit airflow into the prosthetic system 100 and thereby reduce a likelihood of the residual limb of the user overheating. The polygon extrusions 360 may permit airflow into the prosthetic system 100 without including a rigidity of the frame 300.

A gap may be formed at the proximal end 310 of the frame 300 between opposing sides 370a, 370b. Further, the frame 300 may include a slit 380. The frame slit 380 may run from a distal end of the open portion 330 and substantially towards the distal end 320 of the frame 300. As such, if the slit 380 is urged to widen there may be a corresponding widening of the gap formed between the opposing sides 370a, 370b. On the other hand, if the slit 380 is urged to narrow there may be a corresponding narrowing of the gap formed between the opposing sides 370a, 370b. Forcing a change in the width of the slit 380 of the frame 300 may therefore result in a corresponding change in width between the opposing sides 370a, 370b at the proximal end 310 of the frame 300. This, in turn, enables a user to narrow or widen a width of the open portion 330 of the frame 300. As such, a user is able to tighten the frame 300 about the socket 200 or loosen the frame 300 about the socket 200. This configuration may therefore improve an adjustability of the prosthetic system 100.

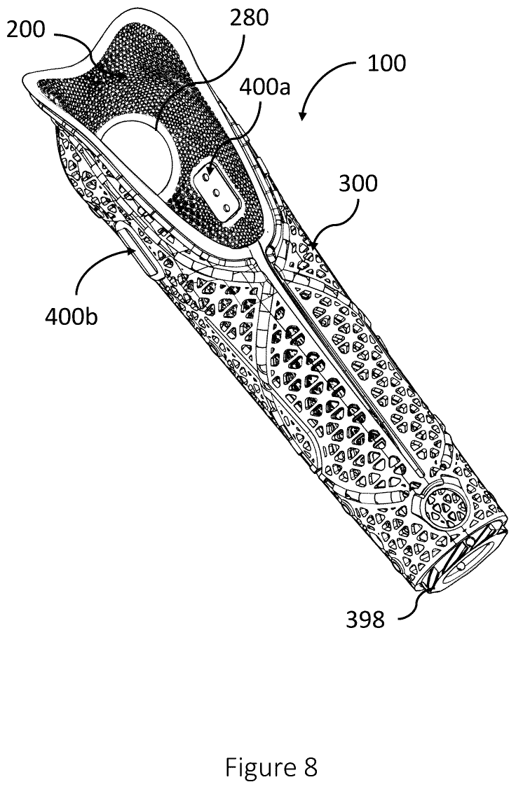

As best shown in FIG. 7, the frame 300 may include a channel 390 configured to receive a cable or lace. In a specific example, the outer surface 350 of the frame 300 may include a channel 390 configured to receive a cable or lace. Alternatively, the inner surface 340 of the frame 300 may include a channel configured to receive a cable or lace. In yet another example, a channel configured to receive a cable or lace may be disposed between the outer surface 350 of the frame 300 and the inner surface 340 of the frame 300. The channel 390 may be shaped such that the lace, once placed in the channel, traces a figure-of-eight like path over the frame 300. The figure-of-eight like path traced by the channel 390 may be such that the point at which the lace crosses-over is substantially towards the proximal end of the frame slit 380. As shown in FIG. 1 and FIG. 8, the frame slit 380 is configured to disposed substantially over the socket slit 260 when the socket 200 is inserted into the frame 300. As such, the lace may be tightened to cause the frame slit 380 to narrow and to cause the socket slit 260 to also narrow. The lace may also be forced to urge the frame slit 380 to widen, which may cause the socket slit 260 to also widen. Alternatively, the lace may be configured to loosen rather than to force the frame slit 380 to widen. In this alternative example, the frame slit 380 may be permitted to widen when acted upon by an external force. For example, loosening of the lace may permit the frame slit 380 to widen when a user inserts their arm into the socket 200. As such, the user is given an increased control over the tightness of the frame 300 around the socket 200. A tensioner may be coupled to an engagement hole 392 disposed towards the distal end 320 of the frame 300. The channel 390 may be configured to converge upon the engagement hole 392. As such, the lace may couple to the tensioner coupled to the engagement hole 392. The tensioner may be a dial system, wherein turning the dial in one direction causes the lace to urge the frame 300 to tighten about the socket 200, and turning the dial in the opposite direction causes the lace to urge the frame 300 to loosen about the socket 200. The user may be protected by any pinching or rubbing that may be caused by any widening or narrowing of the frame slit 380 by the tongue 270 of the socket 200.

Referring to FIG. 7 and FIG. 8, the frame 300 includes frame windows 394a, 394b. Frame window 394a and frame window 394b are configured to be aligned with socket window 290a and socket window 290b respectively when the socket 200 is inserted into the frame 300. As such, the user may be permitted to insert, remove, and adjust each sensor 400 inserted in a socket window 290 when the socket 200 is disposed within the frame 300. Although the example of the present disclosure shows two frame windows 394a, 394b, it is noted that the frame 300 may include one or more frame windows 394, to which each of the one or more socket windows 290 may be aligned with a corresponding frame window 394 when the socket 200 is inserted to the frame 300. Each frame window 394a, 394b may be substantially similar in shape to the lip 292 of the socket window 290. As such, the lip 292 of each socket window may be configured to abut a surface of a corresponding frame window 394. Therefore, aligning a frame window 394 to a socket window 290 may cause the lip 292 of the socket window 290 to mate with the corresponding frame window 394. This may improve a functionality and an ergonomics of the prosthetic system 100 as the socket 200 is stopped from moving relative to the frame 300 when the socket 200 is inserted to the frame 300 and the frame window 394 and the socket window 290 are aligned.

Further, frame windows 394a, 394b may include at least one window tooth 291 configured to engage a corresponding sensor tooth 410 when the sensor 400 is placed into the frame window 394.

As is also shown in FIG. 7 and FIG. 8, a frame through hole 396 may be formed substantially towards the proximal end 310 of the frame 300. The frame through hole 396 may be formed at a distal end of the gap formed between the opposing sides 370a, 370b. The frame through hole 396 may be substantially similar in size and shape to the socket through hole 280. As such, and as best shown in FIG. 8, when the socket 200 is inserted to the frame 300, air flow is permitted to pass through the prosthetic system via the frame through hole 396 and the socket through hole 280.

Still referring to FIG. 7 and FIG. 8, the outer surface 350 of the frame 300 may include distinct portions, wherein each portion may be configured to couple with a corresponding auxiliary module. For example, channel 390 may be configured to couple with a lace, and engagement hole 392 may be configured to couple with a lace tensioning system. There may further be a prosthetic coupling portion 398 formed at the distal end 320 of the frame 300. The prosthetic coupling portion 398 may be configured to couple to a wrist module. The prosthetic coupling portion 398 may alternatively be configured to couple to any other auxiliary module.

The frame 300 may be manufactured by an additive manufacturing process. For example, the frame 300 may be 3D printed. More specifically, the frame 300 may be manufactured using a Multi Jet Fusion 3D printing process. The frame 300 may alternatively be manufactured using any other suitable molding process (e.g., plastic injection molding). The frame 300 may be rigid relative to the socket 200. The frame 300 may be manufactured from nylon. The frame 300 may be manufactured from PLA, a biodegradable thermoplastic. Alternatively, the frame 300 may be manufactured from any material which is rigid relative to the socket 200.

Variations to the disclosed embodiments can be understood and effected by those skilled in the art in practicing the principles and techniques described herein, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored or distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

The principle and mode of operation of this invention have been explained and illustrated in its preferred embodiment. However, it must be understood that this invention may be practiced otherwise than as specifically explained and illustrated without departing from its spirit or scope.

What is claimed is:

1. A prosthetic system comprising:
an inner socket configured to receive a residual limb, the socket comprising at least one socket window; and
an outer frame configured to receive the socket, the frame comprising at least one frame window,
wherein the socket window is configured to align with the frame window when the socket is received within the frame, and
wherein the frame window and the socket window are together configured to releasably receive a sensor for the residual limb.

2. A prosthetic system according to claim 1, wherein the socket window comprises at least one window tooth configured to engage a corresponding sensor tooth when the sensor is placed into the socket window.

3. A prosthetic system according to claim 1, wherein the frame window comprises at least one window tooth configured to engage a corresponding sensor tooth when the sensor is placed into the frame window.

4. A prosthetic system according to claim 2, wherein the socket window and/or frame window comprise a plurality of window teeth, the space between adjacent window teeth being configured to provide a snap-fit for a sensor tooth, such that the sensor tooth is configured to move in sequential snap-fits between adjacent window teeth when a user exerts a force on the sensor.

5. A prosthetic system according to claim 2, wherein the prosthetic system further comprises the sensor, wherein the sensor comprises at least one tooth for engaging the window tooth.

6. A prosthetic system according to claim 2, wherein the sensor is an electromyography sensor.

7. A prosthetic system according to claim 1, wherein the socket comprises a proximal end and a distal end, the proximal end including an open portion configured to allow the residual limb to be inserted into the socket or removed from the socket.

8. A prosthetic system according to claim 7, wherein the socket comprises a socket slit, the socket slit extending from an edge of the open portion and substantially towards the distal end of the socket, and wherein the width of the socket slit at the edge of the open portion is configured to change relative to the width of the socket slit at the end of the socket slit that substantially approaches the distal end of the socket.

9. A prosthetic system according to claim 8, wherein the socket comprises a tongue extending along the length of the socket slit, and wherein the tongue is configured to be disposed adjacent to the residual limb.

10. A prosthetic system according to claim 1, wherein the frame comprises one or more channels configured to receive a cable, the frame further comprising an engagement means configured to receive a cable tensioning device.

11. A prosthetic system according to claim 10, wherein the prosthetic system comprises the cable and the cable tensioning device, the cable being coupled to the cable tensioning device, and wherein operating the cable tensioning device in a first direction urges the frame to tighten about the socket and operating the cable tensioning device in a second direction urges the frame to loosen about the socket.

12. A prosthetic system according to claim 1, wherein the frame comprises a frame proximal end and a frame distal end, the frame proximal end including a frame open portion configured to allow a residual limb to be inserted into the socket or removed from the socket.

13. A prosthetic system according to claim 12, wherein the socket comprises a proximal end and a distal end, the proximal end including an open portion configured to allow the residual limb to be inserted into the socket or removed from the socket, wherein the socket comprises a socket slit, the socket slit extending from an edge of the open portion and substantially towards the distal end of the socket, and wherein the width of the socket slit at the edge of the open portion is configured to change relative to the width of the socket slit at the end of the socket slit that substantially approaches the distal end of the socket, and wherein the frame comprises a frame slit, the frame slit extending from an edge of the frame open portion and substantially towards the frame distal end, the frame slit being configured to be disposed adjacent to the socket slit when the socket is received within the frame.

14. A prosthetic system according to claim 12, wherein the frame comprises one or more channels configured to receive a cable, the frame further comprising an engagement means configured to receive a cable tensioning device, wherein the prosthetic system comprises the cable and the cable tensioning device, the cable being coupled to the cable tensioning device, and wherein operating the cable tensioning device in a first direction urges the frame to tighten about the socket and operating the cable tensioning device in a second direction urges the frame to loosen about the socket, and wherein the frame proximal end comprises a gap, the gap being configured to narrow when the cable tensioning device is operated in the first direction, the gap being configured to widen when the cable tensioning device is operated in the second direction.

15. A prosthetic system according to claim 1, wherein the socket is configured to receive a residual arm limb.
